(19) United States
(12) Patent Application Publication
(10) Pub. No.: US 2010/0111858 A1
(43) Pub. Date: May 6, 2010

(54) Diangostic and Therapeutic Cyclooxygenase-2 Binding Ligands

(76) Inventors: Carol P. Howard, Fenton, MO (US); Dennis A. Moore, St. Louis, MO (US)

Correspondence Address: Mallinckrodt Inc., 675 McDonnell Boulevard, HAZELWOOD, MO, 63042, US

(21) Appl. No.: 12/523,402

(22) PCT Filed: Jan. 17, 2008
(86) PCT No.: PCT/US08/00631

§ 371 Date: Jul. 16, 2009

Related U.S. Application Data
(60) Provisional application No. 60/885,690, filed on Jan. 19, 2007.

(51) Int. Cl. A61K 31/415; C07D 231/12; C07D 403/12; A61K 51/04; A61K 31/4155; A61K 47/48; A61P 35/00

(52) U.S. Cl. 424/1.65; 548/375.1; 534/10; 540/474; 514/406

(57) ABSTRACT

The present invention provides conjugates useful for the diagnosis and treatment of diseases associated with the over-expression of COX-2. The conjugates comprise a selective COX-2 targeting carrier, a metal coordinating moiety, and a linker chemically linking the metal coordinating moiety to the carrier. The metals coordinated by the metal coordinating moiety are selected from paramagnetic or radioisotopes. The invention also includes kits comprising a conjugate and a radioisotope solution.

### BACKGROUND OF THE INVENTION

The present invention is generally directed to metal chelating conjugates for use as a metallopharmaceutical diagnostic or therapeutic agent.

Metallopharmaceutical diagnostic and therapeutic agents are finding ever-increasing application in biological and medical research, and in diagnostic and therapeutic procedures. Generally, these agents contain a radioisotope or paramagnetic metal which, upon introduction to a subject, become localized in a specific organ, tissue or skeletal structure of choice. When the purpose of the procedure is diagnostic, images depicting the in vivo distribution of the radioisotope or paramagnetic metal can be made by various means. The distribution and corresponding relative intensity of the detected radioisotope or paramagnetic metal not only indicates the space occupied by the targeted tissue, but may also indicate a presence of receptors, antigens, aberrations, pathological conditions, and the like. When the purpose of the procedure is therapeutic, the agent typically contains a radioisotope and the radioactive agent delivers a dose of radiation to the local site.

Depending upon the target organ or tissue of interest and the desired diagnostic or therapeutic procedure, a range of metallopharmaceutical agents may be used. One common form is a conjugate comprising a radioactive or paramagnetic metal, a carrier agent for targeting the conjugate to a specific organ or tissue site, and a linkage for chemically linking the metal to the carrier. In such conjugates, the metal is typically associated with the conjugate in the form of a coordination complex, more typically as a chelate of a macrocycle. See, e.g., Liu, U.S. Pat. No. 6,916,460.

### SUMMARY OF THE INVENTION

Among the several aspects of the present invention is the provision of a conjugate for use in diagnostic and therapeutic procedures. Advantageously, such conjugates tend to accumulate in the specific organ, tissue or skeletal structure expressing cyclooxygenase-2 (COX-2) with a reduced risk of non-specific binding to non-target tissues. In diseases that result in the over-expression of COX-2 relative to normal levels of expression, a greater quantity of conjugates bind to the tissues and organs that over-express COX-2 than tissues and organs what express normal levels of COX-2. Thus, a diagnosis of the presence of a disease can be made by identifying a location having a greater concentration of binding relative to normal tissues. Furthermore, in diseases associated with the over-expression of COX-2 which respond to radiotherapy, conjugates having a therapeutic radioisotope can be administered to a patient, the conjugates selectively binding to the disease tissues or organs and provide a localized dose of radiation.

Briefly, therefore, the present invention is directed to a conjugate, the conjugate comprising a carrier for targeting the conjugate to a biological tissue or organ expressing COX-2, a metal coordinating moiety, and a linker chemically linking the metal coordinating moiety to the carrier.

The present invention is further directed to a method for the diagnosis or treatment of cancer or other disease associated with the over-expression of COX-2. The method comprises administering a conjugate to a subject, the conjugate comprising a selective COX-2 targeting carrier for targeting the conjugate to a biological tissue or organ expressing COX-2, a metal coordinating moiety, a radioactive or paramagnetic metal complexed by the metal coordinating moiety, and a linker chemically linking the metal coordinating moiety to the carrier. The conjugate binds to a site of COX-2 over-expression and the cancer is diagnosed or receives a therapeutic amount of radiation.

The present invention is further directed to a kit for the preparation of a metallopharmaceutical. The kit comprises a conjugate for use in a diagnostic or therapeutic method for the detection or treatment of cancer, the conjugate comprising a carrier for targeting the conjugate to a biological tissue or organ over-expressing COX-2, a metal coordinating moiety, a metal complexed by the metal coordinating moiety, and a linker chemically linking the metal coordinating moiety to the carrier.

Another aspect of the present invention includes a method of treating a tumor associated with the expression of prostaglandins. The method comprises administering to a patient an amount of a conjugate comprising a selective COX 2 targeting carrier linked to a metal coordinating moiety chelating a radioisotope. The selective COX-2 targeting carrier binding to a tumor site and reducing the expression of COX-2-derived prostaglandins, wherein the reduction of COX-2-derived prostaglandin expression from administration of the conjugate is greater than the reduction of COX-2-derived prostaglandin expression resulting from the administration of a combination therapy of a non-conjugated COX-2 inhibitor and external radiotherapy.

Other aspects of the invention will be in part apparent and in part pointed out hereinafter.

### DETAILED DESCRIPTION OF THE PREFERRED EMBODIMENTS

The present invention provides conjugates that can rapidly form coordination complexes with metals for use in diagnostic or therapeutic metalloradiopharmaceuticals, or magnetic resonance imaging contrast agents. The conjugates can also serve as bifunctional chelators (BFC's) for attaching metal ions to selective COX-2 targeting carriers, sometimes referred to as biomolecules, which bind in vivo to a tissue type or organ expressing COX-2. The target-specific metallopharmaceuticals of the present invention are useful, for example, in the diagnosis of cancer or other diseases characterized by the over-expression of COX-2 relative to normal tissues by magnetic resonance imaging or scintigraphy.

Generally, the conjugates of the present invention comprise a selective COX-2 targeting carrier and a metal coordinating moiety covalently joined, directly or indirectly, to a linking group. Similarly and independently, the linking group may also be directly bonded to the metal coordinating moiety, or indirectly bonded to the metal coordinating moiety through a series of atoms.

Schematically, a conjugate comprising the biodirecting carrier, a linker, and the metal coordinating moiety of the present invention corresponds to Formula A:

(COX-2)-L-Metal Coordinating Moiety (A)

wherein:

COX-2 is a selective COX-2 targeting carrier,

L is a linker, and

Metal Coordinating Moiety is a moiety that coordinates a radioisotope or paramagnetic metal under physiological conditions.

In combination, the linker covalently links the selective COX-2 targeting carrier to the metal coordinating moiety.

Prior to use in a use in diagnostic and therapeutic procedure, a conjugate corresponding to Formula A is complexed with a metal to form a metallopharmaceutical diagnostic or therapeutic agent of the present invention.

The conjugate can be administered to a patient in a diagnostic or therapeutic procedure. The COX-2 targeting carrier binds to tissues or organs that express COX-2. Once bound, the patient can be imaged to determine the localized concentrations of either paramagnetic or radioisotope metals in the patient. An increased concentration relative to normal or healthy tissues is indicative of COX-2 over-expression and may be indicative of the presence and location of a disease state, e.g., a cancerous tumor. Furthermore, by observing the relative size of the area having a greater relative quantity of bound conjugate, a physician can determine the relative size and shape of a cancerous tumor or diseased tissue or organ. Examples of diseases that can be diagnosed or treated with conjugates of the present invention include, but are not limited to, cancers, for example, bone cancer, brain cancer, breast cancer, colon cancer, liver cancer, lung cancer, pancreatic cancer, prostate cancer, stomach cancer, and thyroid cancer; other diseases associated with the over-expression of COX-2.

Once a patient has been diagnosed to have a cancer or other disease associated with the over-expression of COX-2, the patient can be treated by administering a conjugate of the present invention wherein a therapeutic radioisotope is coordinated to the metal coordinating moiety. Similar to the diagnostic conjugate, the COX-2 targeting carrier binds to tissues or organs that express COX-2. Since the conjugate binds to tissues or organs that are over-expressing COX-2 in greater quantities than normal tissues, a localized therapeutic dose of radiation is administered to the cancerous or diseased site.

Selective COX-2 Targeting Carriers

As previously noted, conjugates of the present invention include selective COX-2 targeting carriers, also known as biomolecules, that direct the conjugate to the targeted tissue or organ that express COX-2. Presently preferred selective COX-2 targeting carriers include COX-2 inhibitors that are approved for pharmaceutical use in humans by regulatory agencies responsible for reviewing and approving the use of pharmaceutical drugs in a given country. For example, preferred COX-2 inhibitors for use as selective COX-2 targeting carriers in the conjugates of the present invention in the United States would be COX-2 inhibitors approved by the Food and Drug Administration (FDA). Preferred COX-2 inhibitors for use as selective COX-2 targeting carriers in conjugates used in Europe would be COX-2 inhibitors approved by the European Medicinal Evaluation Agency (EMEA) for pharmaceutical use in humans.

In one embodiment, the selective COX-2 targeting carrier is a tricyclic COX-2 inhibitor having Formula (B):

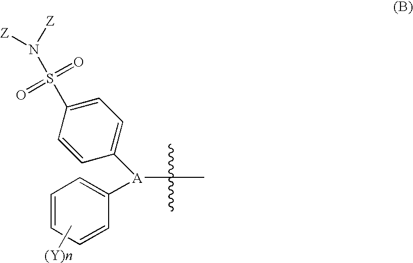

wherein A is a five- or six-membered ring,

each Z is independently H, lower alkyl, hydroxyl, hydroxylalkyl, and halo,

each Y is independently H, lower alkyl, hydroxyl, alkyloxy, halo, haloalkyl, amino, aminoalkyl, and phenyl, and

n is 0-3.

In particular examples of the selective COX-2 targeting carrier, A is a pyrazolyl or furanone ring to yield substituted pyrazolyl or substituted furanone benzenesulfonamide compounds.

In other examples, the selective COX-2 targeting carrier is a tricyclic COX-2 inhibitor having Formula (B), wherein A is a five- or six-membered ring selected from partially unsaturated or unsaturated heterocyclo and carbocyclic rings, optionally substituted with one or more radicals selected from the group consisting of alkyl, halo, oxo, and alkoxy.

One embodiment of the present invention includes a conjugate corresponding to Formula (C):

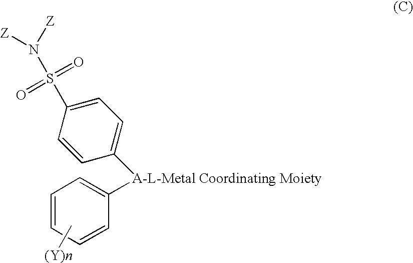

wherein:

A, Y, Z, and n are defined above for the tricyclic COX-2 inhibitor having Formula (B),

Metal Coordinating Moiety is a moiety that coordinates a radioisotope or paramagnetic metal under physiological conditions, and

L is a linker, covalently linking the moiety, A, to the Metal Coordinating Moiety.

One conjugate of the present invention includes celecoxib as the selective COX-2 inhibitor, wherein the conjugate comprising the celecoxib moiety, linker, and metal coordinating moiety corresponds to Formula (D):

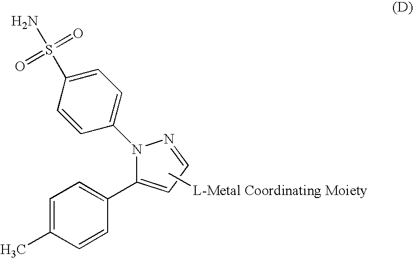

wherein:

Metal Coordinating Moiety is a moiety that coordinates a radioisotope or paramagnetic metal under physiological conditions; and

L is a linker, covalently linking the selective COX-2 targeting carrier to the Metal Coordinating Moiety.

In another embodiment, the selective COX-2 targeting carrier includes a fused polycyclic COX-2 inhibitor having Formula (E):

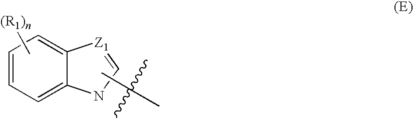

wherein R₁ is lower alkyl, alkoxy, halo, haloalkoxy, or haloalkyl,

n is 0-3, and

Z₁ is carbon or nitrogen, wherein the selective COX-2 targeting carrier is indole when X is carbon and benzimidazole when Z₁ is nitrogen.

One example of a conjugate of the present invention incorporating the fused polycyclic COX-2 inhibitor of Formula (D) corresponds to Formula (F):

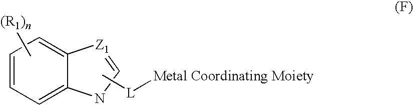

wherein:

R₁, Z₁, and n are defined above for the fused polycyclic COX-2 inhibitor having Formula (D),

Metal Coordinating Moiety is a moiety that coordinates a radioisotope or paramagnetic metal under physiological conditions, and

L is a linker, covalently linking the selective COX-2 targeting carrier to the Metal Coordinating Moiety.

In another embodiment, the selective COX-2 targeting carrier is a fused polycyclic COX-2 inhibitor having Formulas (G) or (H):

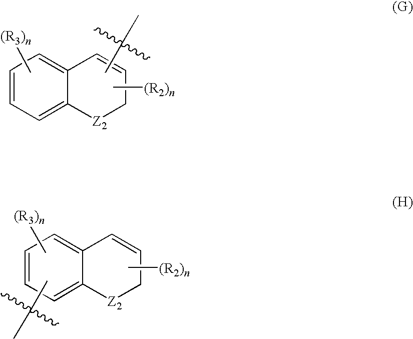

wherein:

R₂ is H, lower alkyl, halo, haloalkyl, alkylthio, alkoxy, arylalkyl, cycloalkyl, phenyl, or alkylsulfonyl,

R₃ is H, lower alkyl, haloalkyl, alkoxy, alkylamino, aryl, arylalkyl, aryloxy, arylamino, nitro, sulfonamide, or carboxamido,

n is 0-3, and

Z₂ is O, S, NR₄, or CR₅R₆, wherein R₄ is H, lower-alkyl, aryl, alkylcarboxylic acid, arylcarboxylic acid, alkylsulfonyl, arylsulfinyl, arylsulfonyl, or sulfonamide, and R₅ and R₆ are each independently H, lower alkyl, lower alkyl-phenyl, haloalkyl, halo, or alkenyl.

Examples of conjugates of the present invention incorporating the fused polycyclic COX-2 inhibitor of Formulas (G) and (H) correspond to Formulas (I) and (J):

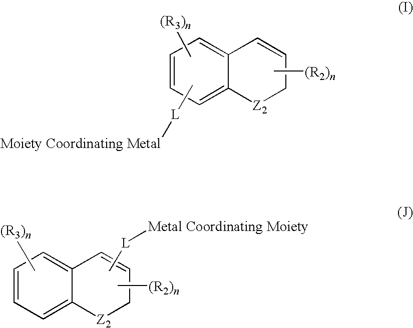

wherein:

R₂, R₃, R₄, R₅, R₆, Z₂, and n are defined above for the fused polycyclic COX-2 inhibitor having Formula (F),

Metal Coordinating Moiety is a moiety that coordinates a radioisotope or paramagnetic metal under physiological conditions, and

L is a linker, covalently linking the selective COX-2 targeting carrier to the Metal Coordinating Moiety.

When Z₂ is O, the selective COX-2 targeting carrier is a benzopyran; when Z₂ is S, the selective COX-2 targeting carrier is a benzothiopyran; when Z₂ is N, the selective COX-2 targeting carrier is a quinoline; and when Z₂ is C, the selective COX-2 targeting carrier is a naphthyl.

Other examples of COX-2 targeting carriers include conjugates derived from selective COX-2 inhibitors such as celecoxib (i.e., 4-[5-(4-methylphenyl)-3-(trifluoromethyl)-1H-pyrazol-1-yl]benzenesulfonamide); cimicoxib (i.e., 4-(4-cyclohexyl-2-methyloxazol-5-yl)-2-fluorobenzenesulfonamide); deracoxib (i.e., 4-[3-(difluoromethyl)-5-(3-fluoro-4-methoxyphenyl)-1H-pyrazol-1-yl]benzenesulfonamide); valdecoxib (i.e., 4-(5-methyl-3-phenyl-4-isoxazolyl)benzenesulfonamide); rofecoxib (i.e., 4-[4-(methylsulfonyl)phenyl]-3-phenyl-2(5H)-furanone); etoricoxib (i.e., 2,3′-bipyridine, 5-chloro-6′-methyl-3-[4-[methylsulfonyl]phenyl]; or [2]5-chloro-6′-methyl-3-[p-[methylsulfonyl]phenyl]-2,3′-bipyridine); meloxicam (i.e., 4-hydroxy-2-methyl-N-(5-methyl-2-thiazolyl)-2H-1,2-benzothiazine-3-carboxamide-1,1-dioxide); parecoxib (i.e., N-[[p-(5-methyl-3-phenyl-4-isoxazolyl)phenyl]sulfonyl]propionamide); 4-(4-cyclohexyl-2-methyloxazol-5-yl)-2-fluorobenzenesulfonamide; 2-(3,5-difluorophenyl)-3-(4-(methylsulfonyl)phenyl)-2-cyclopentene-1-one; N-[2-(cyclohexyloxy)-4-nitrophenyl]methanesulfonamide; 2-(3,4-difluorophenyl)-4-(3-hydroxy-3-methylbutoxy)-5-[4-(methylsulfonyl)phenyl]-3(2H)-pyridazinone; 2-[(2,4-dichloro-6-methylphenyl)amino]-5-ethyl-benzeneacetic acid; (3Z)-3-[(4-chlorophenyl)[4-(methylsulfonyl)phenyl]methylene]-dihydro-2(3H)-furanone; (S)-6,8-dichloro-2-(trifluoromethyl)-2H-1-benzopyran-3-carboxylic acid; lumiracoxib (i.e., [2-[(2-chloro-6-fluorophenyl)amino]-5-methylphenyl]acetic acid); or any pharmaceutically acceptable salts, esters, or prodrugs thereof.

The conjugates of the present invention also include conjugates that incorporate selective COX-2 inhibitors known in the art. Selective COX-2 inhibitors are disclosed in, for example, U.S. Pat. Nos. 5,681,842, 5,750,558, 5,756,531, 5,776,984 and in WO 97/41100, WO 98/39330, WO 99/10331, WO 99/10332 and WO 00/24719 assigned to Abbott Laboratories; and in WO 98/50075, WO 00/29022 and WO 00/29023 assigned to Algos Pharmaceutical Corporation; and in WO 99/15205 assigned to Almirall Prodesfarma S.A.; and in U.S. Pat. No. 5,980,905 assigned to AMBI Inc.; and in U.S. Pat. No. 5,945,538 assigned to American Cyanamid Company; and in U.S. Pat. Nos. 5,776,967, 5,824,699, 5,830,911 and in WO 98/04527 and WO 98/21195 assigned to American Home Products Corporation; and in WO 98/22442 assigned to Angelini Richerche S.P.A. Societa Consortile; and in U.S. Pat. No. 6,046,191 and in WO 99/18960 and WO 00/00200 assigned to Astra Pharmaceuticals Ltd.; and in U.S. Pat. No. 5,905,089 assigned to Board of Supervisors of Louisiana State University; and in WO 97/13767 assigned to Chemisch Pharmazeutische Forschungsgesellschaft MBH; and in WO 98/57924 and WO 99/61436 assigned to Chugai Seiyaku Kabushiki Kaisha; and in WO 00/13685 assigned to Cornell Research Foundation Inc.; and in WO 96/10021 assigned to The Du Pont Merck Pharmaceutical Company; and in EP 0 087 629 B1 assigned to E.I. Du Pont de Nemours and Company; and in WO 99/13799 assigned to Euro-Celtique; and in U.S. Pat. No. 5,134,142 and in WO 91/19708, WO 97/13755, WO 99/15505, WO 99/25695 and in EP 0 418 845 B1 and EP 0 554 829 A2 assigned to Fujisawa Pharmaceutical Co. Ltd.; and in U.S. Pat. Nos. 5,344,991, 5,393,790, 5,434,178, 5,466,823, 5,486,534, 5,504,215, 5,508,426, 5,510,496, 5,516,907, 5,521,207, 5,563,165, 5,580,985, 5,596,008, 5,616,601, 5,620,999, 5,633,272, 5,643,933, 5,668,161, 5,686,470, 5,696,143, 5,700,816, 5,719,163, 5,753,688, 5,756,530, 5,760,068, 5,859,257, 5,908,852, 5,935,990, 5,972,986, 5,985,902, 5,990,148, 6,025,353, 6,028,072, 6,136,839 and in WO 94/15932, WO 94/27980, WO 95/11883, WO 95/15315, WO 95/15316, WO 95/15317, WO 95/15318, WO 95/21817, WO 95/30652, WO 95/30656, WO 96/03392, WO 96/03385, WO 96/03387, WO 96/03388, WO 96/09293, WO 96/09304, WO 96/16934, WO 96/25405, WO 96/24584, WO 96/24585, WO 96/36617, WO 96/38418, WO 96/38442, WO 96/41626, WO 96/41645, WO 97/11704, WO 97/27181, WO 97/29776, WO 97/38986, WO 98/06708, WO 98/43649, WO 98/47509, WO 98/47890, WO 98/52937, WO 99/22720, WO 00/23433, WO 00/37107, WO 00/38730, WO 00/38786 and WO 00/53149 assigned to G. D. Searle & Co.; and in WO 96/31509, WO 99/12930, WO 00/26216 and WO 00/52008 assigned to Glaxo Group Limited; and in EP 1 006 114 A1 and in WO 98/46594 assigned to Grelan Pharmaceutical Co. Ltd.; and in WO 97/34882 assigned to Grupo Farmaceufico Almirall; and in WO 97/03953 assigned to Hafslund Nycomed Pharma AG; and in WO 98/32732 assigned to Hoffman-La Roche AG; and in U.S. Pat. Nos. 5,945,539, 5,994,381, 6,002,014 and in WO 96/19462, WO 96/19463 and in EP 0 745 596 A1 assigned to Japan Tobacco, Inc.; and in U.S. Pat. Nos. 5,686,460, 5,807,873 and in WO 97/37984, WO 98/05639, WO 98/11080 and WO 99/21585 assigned to Laboratories USPA; and in WO 99/62884 assigned to Laboratories Del Dr. Esteve, S.A.; and in WO 00/08024 assigned to Laboratorios S.A.L.V.A.T., S.A.; and in U.S. Pat. Nos. 5,585,504, 5,840,924, 5,883,267, 5,925,631, 6,001,843, 6,080,876 and in WO 97/44027, WO 97/44028, WO 97/45420, WO 98/00416, WO 98/47871, WO 99/15503, WO 99/15513, WO 99/20110, WO 99/45913, WO 99/55830, WO 00/25779 and WO 00/27382 assigned to Merck & Co. Inc.; and in U.S. Pat. Nos. 5,409,944, 5,436,265, 5,474,995, 5,536,752, 5,550,142, 5,510,368, 5,521,213, 5,552,422, 5,604,253, 5,604,260, 5,639,780, 5,677,318, 5,691,374, 5,698,584, 5,710,140, 5,733,909, 5,789,413, 5,817,700, 5,840,746, 5,849,943, 5,861,419, 5,981,576, 5,994,379, 6,020,343, 6,071,936, 6,071,954 and in EP 0 788 476 B1, EP 0 863 134 A1, EP 0 882 016 B1 and in WO 94/20480, WO 94/13635, WO 94/26731, WO 95/00501, WO 95/18799, WO 96/06840, WO 96/13483, WO 96/19469, WO 96/21667, WO 96/23786, WO 96/36623, WO 96/37467, WO 96/37468, WO 96/37469, WO 97/14691, WO 97/16435, WO 97/28120, WO 97/28121, WO 97/36863, WO 98/03484, WO 98/41511, WO 98/41516, WO 98/43966, WO 99/14194, WO 99/14195, WO 99/23087, WO 99/41224 and WO 00/68215 assigned to Merck Frosst Canada & Co., and in WO 99/59635 assigned to Merck Sharp & Dohme Limited; and in U.S. Pat. No. 5,380,738 assigned to Monsanto Company; and in WO 00/01380 assigned to A. Nattermann & Co.; and in WO 99/61016 assigned to Nippon Shinyaku Co. Ltd.; and in WO 99/33796 assigned to Nissin Food Products Co. Ltd.; and in WO 99/11605 assigned to Novartis A G; and in WO 98/33769 assigned to Nycomed Austria GMBH; and in U.S. Pat. Nos. 6,077,869 and 6,083,969 and in WO 00/51685 assigned to Ortho-McNeil Pharmaceutical, Inc.; and in U.S. Pat. No. 5,783,597 assigned to Ortho Pharmaceutical Corporation; and in WO 98/07714 assigned to Oxis International Inc.; and in WO 00/10993 assigned to Pacific Corporation; and in EP 0 937 722 A1 and in WO 98/50033, WO 99/05104, WO 99/35130 and WO 99/64415 assigned to Pfizer Inc.; and in WO 00/48583 assigned to Pozen Inc.; and in U.S. Pat. No. 5,908,858 assigned to Sankyo Company Limited; and in WO 97/25045 assigned to SmithKline Beecham Corporation; and in U.S. Pat. No. 5,399,357 assigned to Takeda Chemical Industries, Ltd.; and in WO 99/20589 assigned to The University of Sydney; and in U.S. Pat. No. 5,475,021 and WO 00/40087 assigned to Vanderbilt University; and in WO 99/59634 assigned to Wakamoto Pharmaceutical Co. Ltd., the disclosures of each of which are incorporated by reference herein in their entirety.

Linker

As previously noted, the selective COX-2 targeting carrier is covalently bonded to the metal coordinating moiety via a linker. The linker can be comprised of a single atom, a chain of atoms, a compound, a polymer, a urea, or any other group that can link the selective COX-2 targeting carrier to the metal coordinating moiety.

Examples of suitable linkers include linkers comprising a hydrocarbyl or substituted hydrocarbyl group.

Examples of polymers include polyalkylene glycols such as polyethylene glycol (PEG), peptides or other polyamino acids.

Examples of other suitable linkers include linkers comprising carbohydrates and cyclodextrins.

One example of a suitable linker of the present invention is shown below, wherein the carbon chain on either end may be shortened or extended in length:

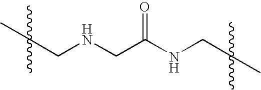

In another example, the linker comprises a urea group.

One example of a linker comprising a urea group corresponds to Formula K:

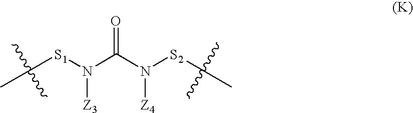

wherein

S₁ and S₂ are independently a covalent bond or a chain of atoms covalently linking the urea moiety to the metal coordinating moiety or bio-directing carrier, respectively; and

Z₃ and Z₄ are independently selected from the group consisting of hydrogen, aryl, C₁₋₇ alkyl, C₁₋₇ hydroxyalkyl and C₁₋₇ alkoxyalkyl. Exemplary Z₃ and Z₄ substituents include hydrogen, C₁₋₇ alkyl, alkoxyalkyl, or phenyl, preferably hydrogen, C₁₋₄ alkyl or C₁₋₄ alkoxyalkyl, and more preferably hydrogen.

In one embodiment, the linker does not contain any amino acid residues.

The linkers are preferably designed to favorably impact biodistribution and potency as well as providing separation between the metal coordinating moiety and the selective COX-2 targeting carrier. For example, the linker may be selected to influence biodistribution of the conjugate, enhance or decrease the rate of blood clearance or direct the route of elimination of the conjugate. In general, preferred linkers are those that result in moderate to fast blood clearance and enhanced renal excretion.

When the linker comprises a chain of atoms, the chain may be linear, branched, cyclic or a combination thereof. In one embodiment, the chain comprises no more than about twenty five atoms. In another embodiment, the chain comprises no more than about fifteen atoms, and in some embodiments, the chain comprises about six to about ten atoms. The atoms comprising this chain are typically selected from the group consisting of carbon, oxygen, nitrogen, sulfur, selenium, silicon and phosphorous. In one embodiment, the group consists of carbon, oxygen, nitrogen, and sulfur. In another embodiment, the group consists of carbon, nitrogen and oxygen.

In one example, the linker is an aryl or C₁₋₂₀ alkylene optionally substituted with one or more carbaldehyde, keto, carboxyl (—CO₂H), cyano (—CN), halo, nitro (—NO₂), amido, sulfato (—OSO₃H), sulfito (—SO₃H), phosphato (—OPO₃H₂), phosphito (—PO₃H₂), hydroxyl (—OH), oxy, mercapto (—SH), and thio (—SO) groups.

In another example, the linker is an aryl optionally substituted with one or more of oxy, keto, halo, and amido, or C₁₋₈ alkylene optionally substituted with one or more oxy and keto, or C₁₋₄ alkylene optionally substituted with oxy.

The linker can also comprise (i) a C₂₋₂₀ alkyl chain or ring optionally substituted with one or more oxygen atoms as ether linkages or pendant with one or more hydroxyl groups as alcohols; (ii) a peptide chain or ring consisting of one or more amino acid residues such as alanine, isoleucine, leucine, valine, phenylalanine, tryptophan, tyrosine, asparagine, methionine, cysteine, serine, glutamine, threonine, aspartic acid, glutamic acid, arginine, histidine, lysine, glycine or proline, conjugated in a natural or unnatural way; and (iii) one or more aromatic rings in chains or condensed in polycycles, optionally substituted with one or more carboxyl, cyano, nitro, amido, hydroxyl, amino, sulfito, phosphito, sulfato, phosphate, C₁₋₂₀ alkyl chain or ring optionally substituted with one or more carboxyl, cyano, nitro, amido, hydroxyl, amino, sulfito, phosphito, sulfato, and phosphate.

Metals

Any metal capable of being detected in a diagnostic procedure in vivo or in vitro or useful in the therapeutic treatment of disease can be employed as a metal in the present conjugates. Particularly, any radioactive metal ion or paramagnetic metal ion capable of producing a diagnostic result or therapeutic response in a human or animal body or in an in vitro diagnostic assay may be used. The selection of an appropriate metal based on the intended purpose is known by those skilled in the art. Typically, the paramagnetic or radioisotope metal is selected from the group consisting of Cr(III), Mn(II), Fe(III), Fe(II), Co(II), Ni(II), Cu(II), Nd(III), Sm(III), Y(III), Gd(III), V(II), Tb(III), Dy(III), Ho(III), Er(III), Cu, Cu-62, Cu-64, Cu-67, Ga, Ga-67, Ga-68, As, As-77, Y, Y-86, Zr-89, Y-90, Tc, Tc═O, Tc-94, Tc-94m, Tc-99m, Tc-99m=O, Pd, Pd-103, In, In-111, Ag-111, I-123, I-124, I-125, I-131, Pr-142, Pm, Pm-149, Gd, Gd-153, Sm, Sm-153, Tb-161, Dy, Dy-165, Dy-166, Ho, Ho-166, Eu, Eu-169, Tm, Tm-170, Lu, Lu-177, Re, Re-186, Re-188, Re═O, Re-186=O, Re-188=O, At, At-211, Bi, Bi-212, Bi-212, Bi-213, Pb-212, Ra-223, and Ac-225.

In one particular embodiment, the metal is a radioisotope selected from the group consisting of Cu-62, Cu-64, Cu-67, Ga-67, Ga-68, As-77, Y-86, Zr-89, Y-90, Tc-94, Tc-94m, Tc-99m, Tc-99m=O, Pd-103, In-111, Ag-111, I-123, I-124, I-125, I-131, Pr-142, Pm-149, Gd-153, Sm-153, Tb-161, Dy-165, Dy-166, Ho-166, Eu-169, Tm-170, Lu-177, Re-186, Re-188, Re═O, Re-186=O, Re-188=O, At-211, Bi-212, Bi-212, Bi-213, Pb-212, Ra-223, and Ac-225.

In a particular example, the metal is selected from Y-90, In-111, Tc-99m, Re-186, Re-188, Cu-64, Ga-67, or Lu-177.

In another example, the metal is selected from Y-90, In-111, Tc-99m, Re-188, or Lu-177.

In another embodiment, the metal is a therapeutic radioisotope selected from the group consisting of Cu-64, Cu-67, Ga-67, Y-90, Ag-111, In-111, I-123, I-131, Pr-142, Sm-153, Tb-161, Dy-166, Ho-166, Lu-177, Re-186, Re-188, Re-189, At-211, Pb-212, Bi-212, Bi-213, Ra-223, and Ac-225.

Particular examples of therapeutic radioisotopes include radioisotopes selected from the group consisting of Re-188, Lu-177, and Y-90.

In another embodiment, the metal is a diagnostic metal selected from the group consisting of Cr(III), Mn(II), Fe(III), Fe(II), Co(II), Ni(II), Cu(II), Nd(III), Sm(III), Y(III), Gd(III), V(II), Tb(III), Dy(III), Ho(III), Er(III), Cu-64, Cu-67, Ga-67, Ga-68, Y-86, Zr-89, Tc-94, Tc-94m, Tc-99m, In-111, I-123, I-124, I-125, and I-131.

Particular examples of diagnostic radioisotopes include radioisotopes selected from the group consisting of Tc-99m and In-111.

Metal Coordinating Moiety

The metal coordinating moiety may be any moiety used to complex (also referred to as “coordinate”) one or more metals under physiological conditions. Preferably, the metal coordinating moiety forms a thermodynamically and kinetically stable complex with the metal to keep the complex intact under physiological conditions; otherwise, systemic release of the coordinated metal may result.

In general, the metal coordinating moiety may be acyclic or cyclic. For example, metal coordinating moieties include diacetic amine; diethylenetriaminepentaacetate (DTPA); polycarboxylic acids such as ethylenediaminetetraacetic Acid (EDTA); DCTA; 1,4,7,10-tetraazacyclododecane-1,4,7,10-tetraacetic acid (DOTA); 1,4,7-triazacyclonane-1,4,7-triacetic acid (NOTA); 1,4,8,11-tetraazacyclotetradecane-N,N′,N″,N′″-tetraacetic acid (TETA); or analogs or homologs thereof. To provide greater stability under physiological conditions, however, macrocyclic moieties (e.g., triaza and tetraza macrocycles) are generally preferred. In some embodiments, the macrocyclic metal coordinating moiety is cyclen or tacn.

In another embodiment of the present invention, a conjugate of the present invention comprises celecoxib as a selective COX-2 inhibitor and DTPA as the metal coordinating moiety. In one embodiment, a conjugate of the present invention corresponds to Formula (L):

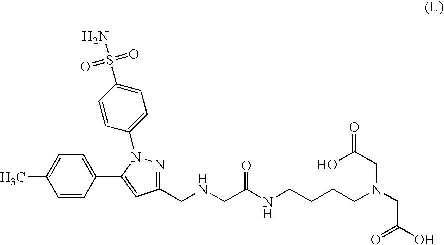

In one embodiment of the present invention, a therapeutic conjugate of the present invention comprises celecoxib as a selective COX-2 targeting carrier, diacetic amine as the metal coordinating moiety, and Re-188 as a diagnostic radioisotope. In one embodiment, a therapeutic conjugate of the present invention corresponds to Formula (M):

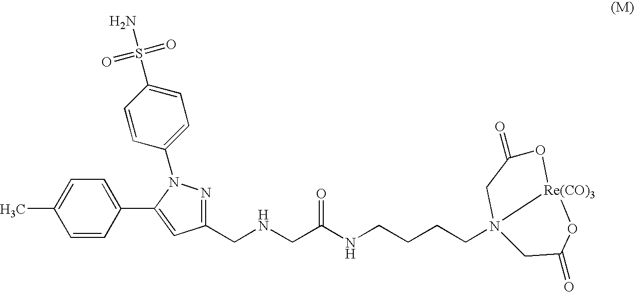

In another embodiment of the present invention, a diagnostic conjugate of the present invention comprises celecoxib as a selective COX-2 targeting carrier, diacetic amine as the metal coordinating moiety, and Tc-99m as a diagnostic radioisotope. In one embodiment, a diagnostic conjugate of the present invention corresponds to Formula (N):

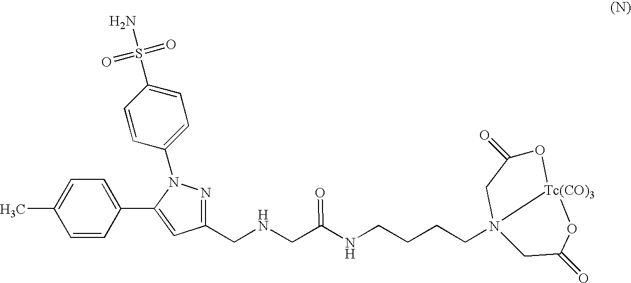

In another embodiment of the present invention, a conjugate of the present invention comprises celecoxib as a selective COX-2 inhibitor and DOTA as the metal coordinating moiety. In one example, a conjugate of the present invention corresponds to Formula (O):

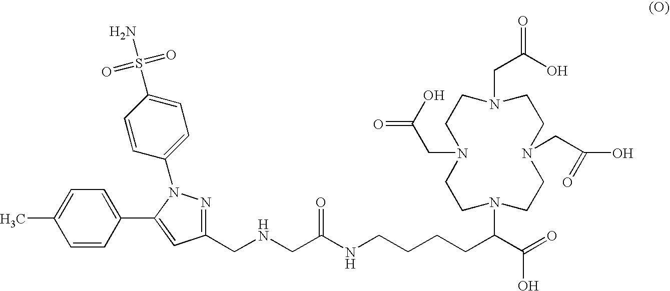

In another embodiment, the metal coordinating moiety comprises a substituted heterocyclic ring where the heteroatom is nitrogen. Typically, the heterocyclic ring comprises from about 9 to about 15 atoms, at least 3 of these ring atoms being nitrogen. Preferably, the heterocyclic ring comprises 3-5 ring nitrogen atoms where at least one of the ring nitrogen atoms is substituted. The ring carbon atoms are optionally substituted. One such preferred macrocycle corresponds to Formula 1:

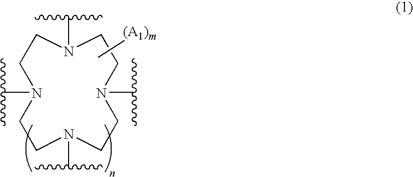

wherein

n is 0, 1 or 2;

m is 0-16 wherein when m is greater than 0, each A₁ is independently selected from the group consisting of optionally substituted C₁₋₂₀ alkyl and aryl.

When the metal coordinating moiety corresponds to Formula 1 and m is greater than zero, it is generally preferred that each A be a substituent that positively impacts stability and biodistribution. When present, each A may independently be substituted with one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio substituents. When A is aryl or alkyl, each of these, in turn, may be optionally substituted with an aryl or C₁₋₂₀ alkyl moiety optionally substituted with one or more aryl, carbaldehyde, keto, carboxyl, cyano, halo, nitro, amido, sulfato, sulfito, phosphato, phosphito, hydroxyl, oxy, mercapto and thio.

For the metal coordinating moieties of Formula (1), the A₁ substituent, if present, is bonded to any of the ring carbon atoms. Further, each ring carbon atom may be substituted so that the number of possible A₁ substituents varies with the number of ring carbon atoms. In a preferred embodiment, each A₁ is independently aryl or C₁₋₈ alkyl optionally substituted with one or more aryl, keto, carboxyl, cyano, nitro, C₁₋₂₀ alkyl, amido, sulfato, sulfito, phosphato, phosphito, oxy and thio; more preferably aryl or C₁₋₆ alkyl optionally substituted with one or more aryl, keto, amido and oxy; and even more preferably methyl.

In general, as the value of n increases, the size of the macrocycle increases. In this manner, the size of the macrocycle may be controlled to match the size and coordination capacity of the metal to be coordinated.

In one embodiment, the metal coordinating moiety comprises a substituted heterocyclic ring, the metal coordinating moiety corresponds to Formula (1a):

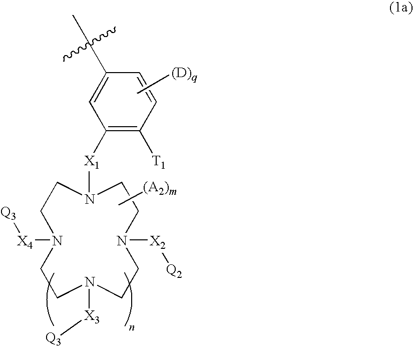

wherein

n is 0, 1 or 2;

m is 0-16, wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;

q is 0-3, wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;

X₁, X₂, X₃, X₄ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;

Q₂-Q₄ are independently selected from the group consisting of:

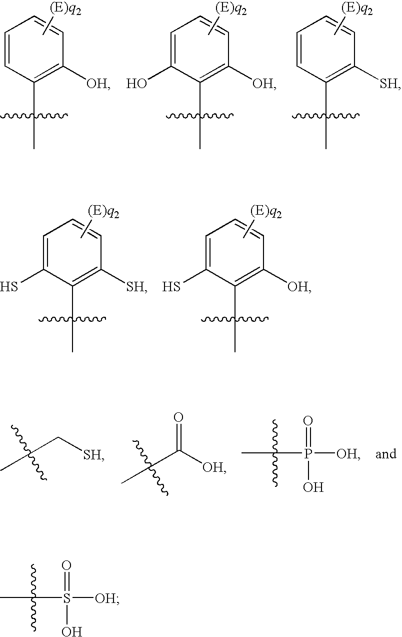

q₂ is 0-4, wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphato; and

T₁ is hydroxyl or mercapto.

For metal coordinating moieties of Formula (1a), the D substituent, if present, is independently bonded to any of the substitutable phenyl ring carbon atoms. In some embodiments, each D may be fluoro, chloro, bromo, iodo, carboxyl, cyano, nitro, amido, hydroxyl, amino, sulfito, phosphito, sulfato, phosphato, aryl, or alkyl optionally substituted with one or more of C₁₋₂₀ alkyl, carboxyl, cyano, nitro, amido, hydroxyl, amino, sulfito, phosphito, sulfato, and phosphate. For example, in some embodiments, each D may be bromo, iodo, carboxyl, or hydroxyl. In some embodiments, when T₁ is hydroxyl, D may be a constituent other than hydroxyl at the position that is alpha to the point of attachment of X₁ and beta to the point of attachment of T₁.

For metal coordinating moieties of Formula (1a), the E substituent, if present, is independently bonded to any of the substitutable phenyl ring carbon atoms. In some embodiments, each E may independently be fluoro, chloro, bromo, iodo, carboxyl, cyano, nitro, amido, hydroxyl, amino, sulfito, phosphito, sulfato, phosphato, aryl, or C₁₋₈ alkyl optionally substituted with one or more of C₁₋₂₀ alkyl, carboxyl, cyano, nitro, amido, hydroxyl, amino, sulfito, phosphito, sulfato, and phosphato. For example, in some embodiments, each E may independently be bromo, iodo, carboxyl, or hydroxyl.

Typically, for metal coordinating moieties of Formula (1a), X₁-X₄ are independently methylene optionally substituted by C₁₋₆ alkyl, halo, or hydroxyl.

In some embodiments of the metal coordinating moieties of Formula (1a), q₂ is 0. Accordingly, Q₂, Q₃, and Q₄ may independently be selected from the group consisting of:

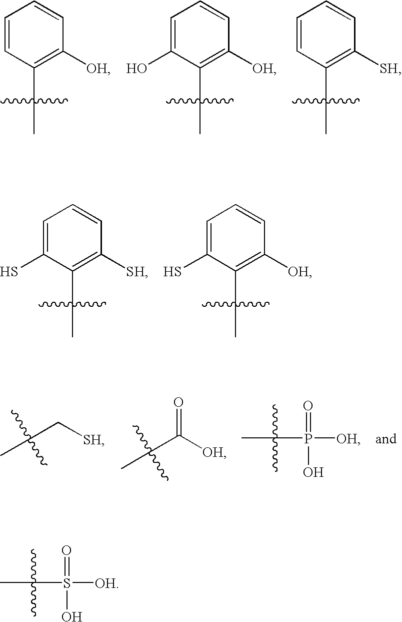

In addition to the metal coordinating moieties including a heterocyclic ring, the metal coordinating moieties may alternatively include a heterosubstituted alkyl chain. Typically, the heterosubstituted alkyl chain includes from about 4 to about 10 atoms in the heterosubstituted alkyl chain, at least 2 of the atoms being nitrogen. In one example of metal coordinating moieties including a heterosubstituted alkyl chain, the chain includes 2-4 nitrogen atoms wherein at least one of the chain nitrogen atoms is substituted. For these embodiments, the chain carbon atoms may optionally be substituted. Typically, the nitrogen atoms including the heterosubstituted alkyl chain are separated from each other by two carbon atoms and thus the metal coordinating moiety may be depicted by the following Formula (2):

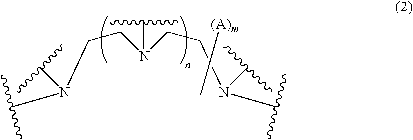

wherein

n is 0, 1 or 2; and

m is 0-8 wherein when m is greater than 0, each A is independently selected from the group consisting of optionally substituted C₁₋₂₀ alkyl and aryl.

When the metal coordinating moiety corresponds to Formula (2) and m is greater than 0, it is generally preferred that each A be a substituent that positively impacts stability and biodistribution. When present, each A may independently be substituted with one or more aryl, C₁₋₂₀ alkyl, carbaldehyde, keto, carboxyl, cyano, halo, nitro, amido, sulfato, sulfito, phosphato, phosphito, hydroxyl, oxy, mercapto, or thio substituents. In addition, when A is aryl or alkyl, each of these, in turn, may be optionally substituted with an aryl or C₁₋₂₀ alkyl moiety optionally substituted with one or more aryl, carbaldehyde, keto, carboxyl, cyano, halo, nitro, amido, sulfato, sulfito, phosphato, phosphito, hydroxyl, oxy, mercapto and thio.

For metal coordinating moieties of Formula (2), the A substituent, if present, may be bonded to any of the ring carbon atoms. Each ring carbon atom may be substituted so that the number of possible A substituents varies with the number of ring carbon atoms. In one embodiment of metal coordinating moieties of Formula (2) having at least one A substituent, each A is independently aryl or C₁₋₈ alkyl optionally substituted with one or more aryl, keto, carboxyl, cyano, nitro, C₁₋₂₀ alkyl, amido, sulfato, sulfito, phosphato, phosphito, oxy and thio. For example, each A may be aryl or C₁₋₆ alkyl optionally substituted with one or more aryl, keto, amido and oxy. By way of further example, each A may be methyl.

In general, as the value of n increases, the length of the heterosubstituted alkyl chain increases. In this manner, the length of the heterosubstituted alkyl chain may be controlled to match the size and coordination capacity of the metal to be coordinated.

In some embodiments where the metal coordinating moiety includes a heterosubstituted alkyl chain, the metal coordinating moiety complies with the following Formula (2a):

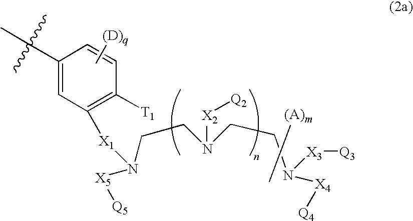

wherein

n is 0, 1 or 2;

m is 0-8 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;

q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;

X₁, X₂, X₃, X₄, and X₅ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;

Q₂-Q₅ are independently selected from the group consisting of:

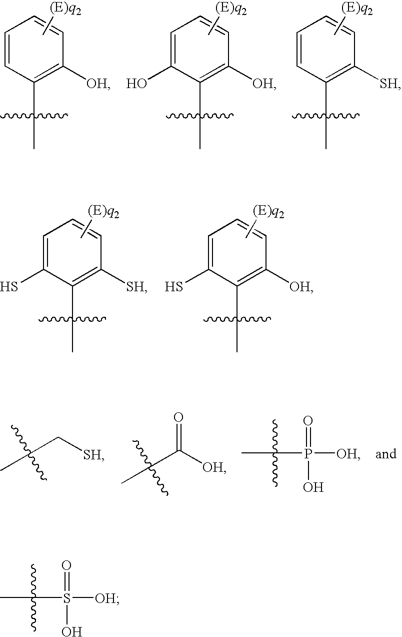

q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphato; and

T₁ is hydroxyl or mercapto.

For metal coordinating moieties of Formula (2a), the D substituent, if present, may be independently bonded to any of the substitutable phenyl ring carbon atoms. In some embodiments, each D may independently be fluoro, chloro, bromo, iodo, carboxyl, cyano, nitro, amido, hydroxyl, amino, sulfito, phosphito, sulfato, phosphato, aryl, or C₁₋₈ alkyl optionally substituted with one or more of C₁₋₂₀ alkyl, carboxyl, cyano, nitro, amido, hydroxyl, amino, sulfito, phosphito, sulfato, and phosphate. For instance, each D of some embodiments may independently be bromo, iodo, carboxyl, or hydroxyl. In some embodiments, when T₁ is hydroxyl, D may be a constituent other than hydroxyl at the position that is alpha to the point of attachment of X₁ and beta to the point of attachment of T₁.

For metal coordinating moieties of Formula (2a), the E substituent, if present, may be independently bonded to any of the substitutable phenyl ring carbon atoms. In some embodiments, each E may independently be fluoro, chloro, bromo, iodo, carboxyl, cyano, nitro, amido, hydroxyl, amino, sulfito, phosphito, sulfato, phosphato, aryl, or C₁₋₈ alkyl optionally substituted with one or more of C₁₋₂₀ alkyl, carboxyl, cyano, nitro, amido, hydroxyl, amino, sulfito, phosphito, sulfato, and phosphato. For instance, each E may independently be bromo, iodo, carboxyl, or hydroxyl in some embodiments.

Typically, for metal coordinating moieties of Formula (2a), X₁-X₄ are independently methylene optionally substituted by C₁₋₆ alkyl, halo, or hydroxyl.

In some embodiments of metal coordinating moieties of Formula (2a), q₂ is 0. Accordingly, Q₂, Q₃, Q₄ and Q₅ are independently selected from the group consisting of:

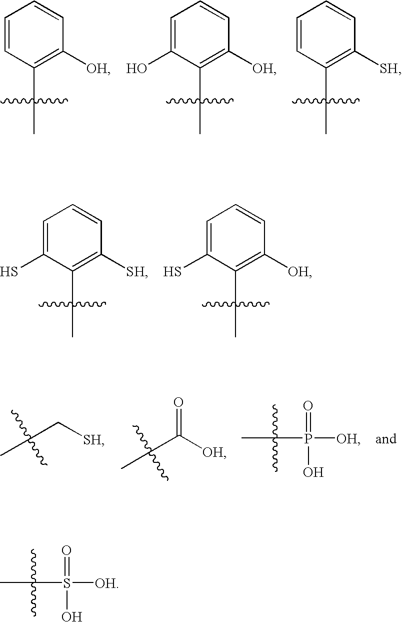

For any of the above embodiments, the metal coordinating moiety may be complexed with a metal, M, thereby forming a metal complex.

In some embodiments where the metal coordinating moiety is a heterocyclic ring and complexed with a metal, M, the complex has the following Formula (3):

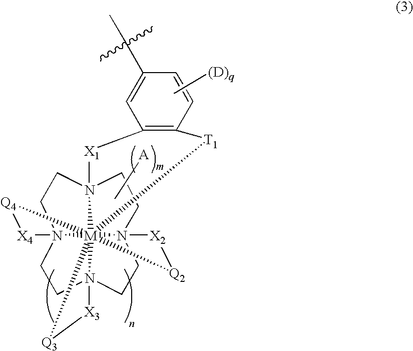

wherein

n is 0, 1 or 2;

m is 0-16 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;

q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;

X₁, X₂, X₃, X₄ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;

Q₂-Q₄ are independently selected from the group consisting of:

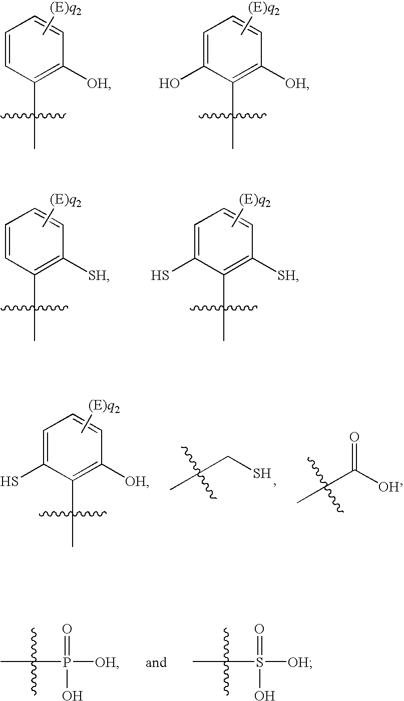

q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphato;

T₁ is hydroxyl or mercapto; and

M is selected from the group consisting of Lu, Lu-177, Y, Y-90, In, In-111, Tc, Tc═O, Tc-99m, Tc-99m=O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

In some embodiments where the metal coordinating moiety is a heterosubstituted alkyl chain and is complexed with a metal, M, the complex has the following Formula (4):

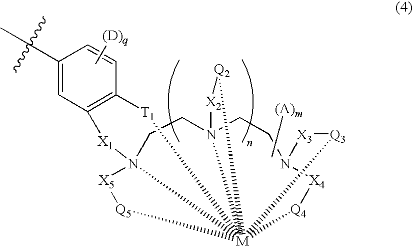

wherein

n is 0, 1 or 2;

m is 0-8 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;

q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;

X₁, X₂, X₃, X₄ and X₅ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;

Q₂-Q₅ are independently selected from the group consisting of:

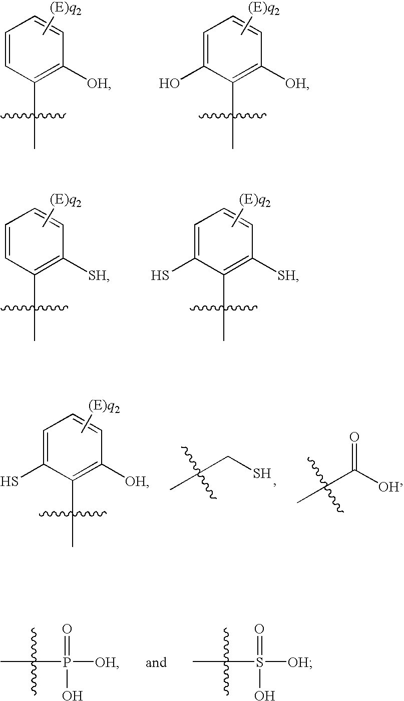

q₂ is 0-4, wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfato, and phosphato;

T₁ is hydroxyl or mercapto; and

M is selected from the group consisting of Lu, Lu-177, Y, Y-90, In, In-111, Tc, Tc═O, Tc-99m, Tc-99m=O, Re, Re-186, Re-188, Re═O, Re-186=O, Re-188=O, Ga, Ga-67, Ga-68, Cu, Cu-62, Cu-64, Cu-67, Gd, Gd-153, Dy, Dy-165, Dy-166, Ho, Ho-166, Eu, Eu-169, Sm, Sm-153, Pd, Pd-103, Pm, Pm-149, Tm, Tm-170, Bi, Bi-212, As and As-211.

Whether the complex corresponds to Formula (3) or Formula (4) typically depends on the particular metal selected for coordination. For example, for yttrium and lanthanides, the complex corresponding to Formula (3) is preferred. Formula (3) is also preferred for iron, copper, and manganese, while Formula (4) is the preferred complex for the remaining transition metals. The preferred complex for any particular metal is related to the potential for transmetallation with endogenous ion. Thus, Formula (3) provides greater stability with high exchange metals, including, but not limited to, yttrium, lanthanides, and gallium. Transmetallation with endogenous ions does not present as great a concern for regular transition metals. While complexes of Formula (3) have been mentioned above as being preferred for use with some metals, while complexes of Formula (4) have been mentioned above as being preferred for use with other metals, it is contemplated that complexes of Formulas (3) and (4) may be utilized with metals other than those listed for the respective complexes.

Macrocyclic metal coordinating moieties with three-dimensional cavities often form metal complexes with high stability. These complexes often exhibit selectivity for certain metal ions based on metal size and coordination chemistry, and capability to adopt a preorganized conformation in the uncomplexed form, which facilitates metal complexation. The selection of appropriate macrocyclic metal coordinating moieties and metals is known by those skilled in the art.

The value of n, and hence the size or length of the metal coordinating moiety, depends upon the particular metal to be coordinated. For yttrium and lanthanides, for example, n is generally 1. For transition metals, n is typically 0 or 1. For manganese and technetium, n is 0, 1, or 2 depending on the value of X₂-X₄. It is, however, contemplated that other values of n may be appropriate for one or more of the metals discussed above.

General Synthesis

For illustrative purposes, the following reaction shows the activation of a metal chelator using carbonyl ditriazine (CDT):

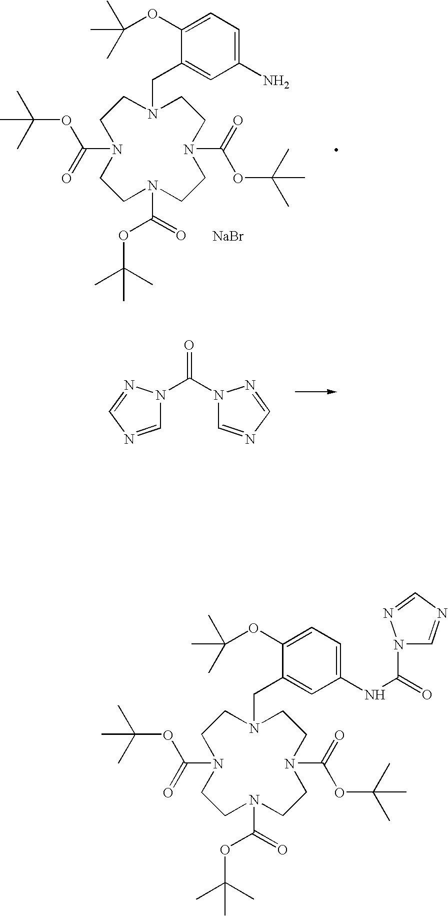

To prevent the reaction of free hydroxyl groups prior to preparation of the conjugate, the hydroxyl groups of the metal coordinating moiety are protected. Any conventional means of protecting the hydroxyl groups is permissible. A variety of protecting groups for the hydroxyl groups and the synthesis thereof may be found in “Protective Groups in Organic Synthesis, 3rd Edition” by T. W. Greene and P. G. M. Wuts, John Wiley and Sons, 1999. Exemplary protecting groups include tert-butyl, methoxymethyl, 1-ethoxymethyl, benzyloxymethyl, (beta-trimethylsilylethoxy)methyl, tetrahydropyranyl, 2,2,2-trichloroethyoxycarbonyl, t-butyl(diphenyl)silyl, trialkylsilyl, trichloromethoxycarbonyl and 2,2,2-trichloroethoxymethyl.

To create a reactive urea group from an amine, a mild activating agent is preferred. Exemplary activating agents include carbonyl ditriazine or carbonyl diimidazole (CDI), or mixtures thereof. Other activating agents include phosgene, bis(trichloromethyl)carbonate, and trichloromethyl chloroformate. The reactive intermediates can be isolated as solids, which are stable while under anhydrous conditions. Thus, such an active urea could be allowed to react with a synthetic or natural product (e.g., a biomolecule) to give a protected intermediate. The product may be isolated by precipitation from the reaction mixture using, for example, dichloromethane and ether. Purification of the product can be carried out, for example, by using normal or C18 reverse phase chromatography, as needed. This intermediate can be subsequently deprotected by application of an acid, such as triflic acid in trifluoroethanol, thereby unmasking the phenol hydroxyl and carboxylates.

For this embodiment, the bio-directing carrier and metal may be any of those previously recited. The radioisotope or paramagnetic metal ion is typically dissolved in a solution. The solution may be an aqueous acid or any other solution known in the art to dissolve a radioisotope or paramagnetic metal ion. The solution should allow for the stable storage of the metal in the kit and not interfere with the properties of the metal. Solubilization aids useful in the preparation of radiopharmaceuticals and in the diagnostic kits include, but are not limited to, ethanol, glycerin, polyethylene glycol, propylene glycol, polyoxyethylene sorbitan monooleate, sorbitan monoloeate, polysorbates, poly(oxyethylene)-poly(oxypropylene)-poly(oxyethylene) block copolymers (Pluronics) and lecithin. Preferred solubilizing aids are polyethylene glycol and Pluronics.

Metallopharmaceutical Compositions

Metallopharmaceutical compositions of the present invention include a conjugate, complexed to a metal, dispersed in a pharmaceutically acceptable carrier. The pharmaceutically acceptable carrier, also known in the art as an excipient, vehicle, auxiliary, adjuvant, or diluent, is typically a substance which is pharmaceutically inert, confers a suitable consistency or form to the composition, and does not diminish the therapeutic or diagnostic efficacy of the conjugate. The carrier is generally considered to be “pharmaceutically or pharmacologically acceptable” if it does not produce an unacceptably adverse, allergic or other untoward reaction when administered to a mammal, especially a human.

The selection of a pharmaceutically acceptable carrier tends, at least in part, to be a function of the desired route of administration. In general, metallopharmaceutical compositions of the invention can be formulated for any route of administration so long as the target tissue is available via that route. For example, suitable routes of administration include, but are not limited to, oral, parenteral (e.g., intravenous, intraarterial, subcutaneous, rectal, subcutaneous, intramuscular, intraorbital, intracapsular, intraspinal, intraperitoneal, or intrasternal), topical (nasal, transdermal, intraocular), intravesical, intrathecal, enteral, pulmonary, intralymphatic, intracavital, vaginal, transurethral, intradermal, aural, intramammary, buccal, orthotopic, intratracheal, intralesional, percutaneous, endoscopical, transmucosal, sublingual and intestinal administration.

Examples of pharmaceutically acceptable carriers for use in compositions of the present invention are well known to those of ordinary skill in the art and may be selected based upon a number of factors: the particular conjugate used, and its concentration, stability and intended bioavailability; the disease, disorder or condition being treated or diagnosed with the composition; the subject, its age, size and general condition; and the route of administration. Suitable nonaqueous, pharmaceutically-acceptable polar solvents include, but are not limited to, alcohols (e.g., α-glycerol formal, β-glycerol formal, 1,3-butyleneglycol, aliphatic or aromatic alcohols having 2-30 carbon atoms such as methanol, ethanol, propanol, isopropanol, butanol, t-butanol, hexanol, octanol, amylene hydrate, benzyl alcohol, glycerin (glycerol), glycol, hexylene glycol, tetrahydrofurfuryl alcohol, lauryl alcohol, cetyl alcohol, or stearyl alcohol, fatty acid esters of fatty alcohols such as polyalkylene glycols (e.g., polypropylene glycol, polyethylene glycol), sorbitan, sucrose and cholesterol); amides (e.g., dimethylacetamide (DMA), benzyl benzoate DMA, dimethylformamide, N-(β-hydroxyethyl)lactamide, N,N-dimethylacetamide amides, 2-pyrrolidinone, 1-methyl-2-pyrrolidinone, or polyvinylpyrrolidone); esters (e.g., 1-methyl-2-pyrrolidinone, 2-pyrrolidinone, acetate esters such as monoacetin, diacetin, and triacetin, aliphatic or aromatic esters such as ethyl caprylate or octanoate, alkyl oleate, benzyl benzoate, benzyl acetate, dimethylsulfoxide (DMSO), esters of glycerin such as mono, di, or tri-glyceryl citrates or tartrates, ethyl benzoate, ethyl acetate, ethyl carbonate, ethyl lactate, ethyl oleate, fatty acid esters of sorbitan, fatty acid derived PEG esters, glyceryl monostearate, glyceride esters such as mono, di, or tri-glycerides, fatty acid esters such as isopropyl myristrate, fatty acid derived PEG esters such as PEG-hydroxyoleate and PEG-hydroxystearate, N-methylpyrrolidinone, pluronic 60, polyoxyethylene sorbitol oleic polyesters such as poly(ethoxylated)₃₀₋₆₀ sorbitol poly(oleate)₂₋₄, poly(oxyethylene)₁₅₋₂₀ monooleate, poly(oxyethylene)₁₅₋₂₀ mono 12-hydroxystearate, and poly(oxyethylene)₁₅₋₂₀ mono ricinoleate, polyoxyethylene sorbitan esters such as polyoxyethylene-sorbitan monooleate, polyoxyethylene-sorbitan monopalmitate, polyoxyethylene-sorbitan monolaurate, polyoxyethylene-sorbitan monostearate, and Polysorbate® 20, 40, 60 or 80 from ICI Americas, Wilmington, Del., polyvinylpyrrolidone, alkyleneoxy modified fatty acid esters such as polyoxyl 40 hydrogenated castor oil and polyoxyethylated castor oils (e.g., Cremophor® EL solution or Cremophor® RH 40 solution), saccharide fatty acid esters (i.e., the condensation product of a monosaccharide (e.g., pentoses such as ribose, ribulose, arabinose, xylose, lyxose and xylulose, hexoses such as glucose, fructose, galactose, mannose and sorbose, trioses, tetroses, heptoses, and octoses), disaccharide (e.g., sucrose, maltose, lactose and trehalose) or oligosaccharide or mixture thereof with a C₄-C₂₂ fatty acid(s) (e.g., saturated fatty acids such as caprylic acid, capric acid, lauric acid, myristic acid, palmitic acid and stearic acid, and unsaturated fatty acids such as palmitoleic acid, oleic acid, elaidic acid, erucic acid and linoleic acid)), or steroidal esters); alkyl, aryl, or cyclic ethers having 2-30 carbon atoms (e.g., diethyl ether, tetrahydrofuran, dimethyl isosorbide, diethylene glycol monoethyl ether); glycofurol (tetrahydrofurfuryl alcohol polyethylene glycol ether); ketones having 3-30 carbon atoms (e.g., acetone, methyl ethyl ketone, methyl isobutyl ketone); aliphatic, cycloaliphatic or aromatic hydrocarbons having 4-30 carbon atoms (e.g., benzene, cyclohexane, dichloromethane, dioxolanes, hexane, n-decane, n-dodecane, n-hexane, sulfolane, tetramethylenesulfon, tetramethylenesulfoxide, toluene, dimethylsulfoxide (DMSO), or tetramethylenesulfoxide); oils of mineral, vegetable, animal, essential or synthetic origin (e.g., mineral oils such as aliphatic or wax-based hydrocarbons, aromatic hydrocarbons, mixed aliphatic and aromatic based hydrocarbons, and refined paraffin oil, vegetable oils such as linseed, tung, safflower, soybean, castor, cottonseed, groundnut, rapeseed, coconut, palm, olive, corn, corn germ, sesame, persic and peanut oil and glycerides such as mono-, di- or triglycerides, animal oils such as fish, marine, sperm, cod-liver, haliver, squalene, squalane, and shark liver oil, oleic oils, and polyoxyethylated castor oil); alkyl or aryl halides having 1-30 carbon atoms and optionally more than one halogen substituent; methylene chloride; monoethanolamine; petroleum benzin; trolamine; omega-3 polyunsaturated fatty acids (e.g., alpha-linolenic acid, eicosapentaenoic acid, docosapentaenoic acid, or docosahexaenoic acid); polyglycol ester of 12-hydroxystearic acid and polyethylene glycol (Solutol® HS-15, from BASF, Ludwigshafen, Germany); polyoxyethylene glycerol; sodium laurate; sodium oleate; or sorbitan monooleate.

Other pharmaceutically acceptable solvents for use in the invention are well known to those of ordinary skill in the art, and are identified in The Chemotherapy Source Book (Williams & Wilkens Publishing), The Handbook of Pharmaceutical Excipients, (American Pharmaceutical Association, Washington, D.C., and The Pharmaceutical Society of Great Britain, London, England, 1968), Modern Pharmaceutics, (G. Banker et al., eds., 3d ed.) (Marcel Dekker, Inc., New York, N.Y., 1995), The Pharmacological Basis of Therapeutics, (Goodman & Gilman, McGraw Hill Publishing), Pharmaceutical Dosage Forms, (H. Lieberman et al., eds.) (Marcel Dekker, Inc., New York, N.Y., 1980), Remington's Pharmaceutical Sciences (A. Gennaro, ed., 19th ed.) (Mack Publishing, Easton, Pa., 1995), The United States Pharmacopeia 24, The National Formulary 19, (National Publishing, Philadelphia, Pa., 2000), A. J. Spiegel et al., and Use of Nonaqueous Solvents in Parenteral Products, Journal of Pharmaceutical Sciences, Vol. 52, No. 10, pp. 917-927 (1963).

Treatment of Tumors Associated with Prostaglandin Synthesis

Conjugates comprising a COX-2 targeting carrier, linker, and metal coordinating moiety of the present invention can be utilized in the treatment of tumors associated with enhanced prostaglandin synthesis. Several types of tumors have been known to express high levels of COX-2 relative to normal tissue. The high COX-2 levels are in turn associated with enhanced expression of prostaglandins. It has been suggested that increased levels of prostaglandins may support and protect tumor growth through the promotion of angiogenesis and in neovasculator formation in tumors. Several varieties of prostaglandins have been identified as being elevated in tumor tissue relative to normal surrounding tissue include prostaglandin E₂ (PGE₂), prostaglandin F_{2α} (PGF_{2α}), 6-Keto-prostaglandin F_{1α} (PGF_{1α}), and thromboxane B₂ (TxB₂).

Without being held to theory, prostaglandin compounds, and PGE₂ in particular, are believed to enhance the survival of tumor cells following ionizing radiotherapy due to their properties of promoting vascular repair and/or angiogenesis in tumor tissue. Ionizing radiation is used in treatment of cancer by damaging the DNA in tumor cells that are rapidly dividing as well as forming free radicals in tissues. The damaging effect of radiation may be observed in increased permeability in tumor tissue neovasculature. The presence of prostaglandins in tumor tissue appears to induce repair of damaged tissue, promotion of neovasculature, and decrease vascular permeability, thereby moderate the effect of radiotherapy. By administering COX-2 inhibitors, the expression of prostaglandins within tumor tissue is reduced. The reduction of prostaglandins in turn results in inhibiting or reducing vascular repair and angiogenesis within tumor tissue, increasing vascular permeability, and improving the effect of radiotherapy on tumor tissues.

In one embodiment, the conjugates comprising a COX-2 targeting carrier, linker, and metal coordinating moiety coordinated to a radiotherapeutic isotope are administered to a patient afflicted with a tumor expressing prostaglandins for treatment and reduction of the tumor. The conjugates of the present invention can be administered to a patient and provide a dual purpose of inhibiting COX-2 expression within a tumor, thereby reducing expression levels of prostaglandins in the tumor, and simultaneously providing localized radiotherapy to a tumor site. The conjugates thus beneficially serve to target a radiotherapeutic isotope to tumor tissues by use of a COX-2 targeting carrier which reduces or inhibits the expression of prostaglandins in the tumor tissues due to the COX-2 inhibiting properties of the COX-2 targeting carrier. The conjugates further beneficially provide localized ionizating radiation to tumor tissue, thereby avoiding excess radiation damage to healthy tissues that can result from external radiotherapy. Examples of radiotherapeutic isotopes that may be coordinated to the conjugate include Cu-64, Cu-67, Ga-67, Y-90, Ag-111, In-111, I-123, I-131, Pr-142, Sm-153, Tb-161, Dy-166, Ho-166, Lu-177, Re-186, Re-188, Re-189, At-211, Pb-212, Bi-212, Bi-213, Ra-223, and Ac-225. The administration of the conjugate coordinating a radiotherapeutic isotope to a patient afflicted with a tumor expressing prostaglandins can result in a greater reduction of the size of the tumor than a combination therapy of administering similar dose of a COX-2 inhibitor monomer corresponding to the COX-2 targeting carrier and a similar dose of externally administered radiotherapy.

In another embodiment, the administration of the conjugates reduces the COX-2 derived prostaglandin expression in tumor tissues by at least about 70% of the pretreatment levels. In another embodiment, the administration of the conjugates reduces the COX-2 derived prostaglandin expression in tumor tissues by at least about 80% of the pretreatment levels. In still another embodiment, the administration of the conjugates reduces the COX-2 derived prostaglandin expression in tumor tissues by at least about 90% of the pretreatment levels.

In another embodiment, the administration to a patient afflicted with a tumor expressing prostaglandins of conjugates comprising a COX-2 targeting carrier, linker, and metal coordinating moiety coordinated to a radiotherapeutic isotope result in increased vascular permeability in the tumor within about a day of administering the conjugate.

Dosage

Dosage and regimens for the administration of the pharmaceutical compositions of the invention can be readily determined by those with ordinary skill in diagnosing or treating disease. It is understood that the dosage of the conjugates will be dependent upon the age, sex, health, and weight of the recipient, kind of concurrent treatment, if any, frequency of treatment, and the nature of the effect desired. For any mode of administration, the actual amount of conjugate delivered, as well as the dosing schedule necessary to achieve the advantageous effects described herein, will also depend, in part, on such factors as the bioavailability of the conjugate, the disorder being treated or diagnosed, the desired therapeutic or diagnostic dose, and other factors that will be apparent to those of skill in the art. The dose administered to an animal, particularly a human, in the context of the present invention should be sufficient to affect the desired therapeutic or diagnostic response in the animal over a reasonable period of time.

Radiolabeled scintigraphic imaging agents provided by the present invention are provided having a suitable amount of radioactivity. In forming diagnostic radioactive complexes, it is generally preferred to form radioactive complexes in solutions containing radioactivity at concentrations of from about 0.01 millicurie (mCi) to about 100 mCi per mL. Generally, the unit dose to be administered has a radioactivity of about 0.01 mCi to about 100 mCi, preferably about 1 mCi to about 30 mCi. The solution to be injected at unit dosage is from about 0.01 mL to about 10 mL. The amount of radiolabeled conjugate appropriate for administration is dependent upon the distribution profile of the chosen conjugate in the sense that a rapidly cleared conjugate may need to be administered in higher doses than one that clears less rapidly. In vivo distribution and localization can be tracked by standard scintigraphic techniques at an appropriate time subsequent to administration; typically between thirty minutes and 180 minutes depending upon the rate of accumulation at the target site with respect to the rate of clearance at the non-target tissue.

Typically, an In-111 diagnostic dose is 3-6 mCi while a typical Tc-99m dose is 10-30 mCi. Generally, radiotherapeutic doses of radiopharmaceuticals vary to a greater extent, depending on the tumor and number of injections of cycles. For example, cumulative doses of Y-90 range from about 100-600 mCi (20-150 mCi/dose), while cumulative doses of Lu-177 range from about 200-800 mCi (50-200 mCi/dose).

Paramagnetic metal imaging agents provided by the present invention are administered to a patient in a dosage suitable for the targeted location and type of image being sought. In one embodiment, a paramagnetic metal contrast agent is administered to a patient in a dosage between about 0.05 and about 0.3 millimoles/kilogram bodyweight. In one example, a gadolinium based contrast agent of the present invention is administered to a patient in a dosage between about 0.1 and about 0.3 millimoles/kilogram bodyweight.

provided having a suitable amount of radioactivity. In forming diagnostic radioactive complexes, it is generally preferred to form radioactive complexes in solutions containing radioactivity at concentrations of from about 0.01 millicurie (mCi) to about 100 mCi per mL. Generally, the unit dose to be administered has a radioactivity of about 0.01 mCi to about 100 mCi, preferably about 1 mCi to about 30 mCi.

Kits

For convenience, metallopharmaceutical compositions of the present invention may be provided to the user in the form of a kit containing some or all of the necessary components. The use of a kit is particularly convenient since some of the components, e.g., a radioisotope, have a limited shelf life, particularly when combined. Thus, the kit may include one or more of the following components (i) a conjugate, (ii) a metal coordinated to or for coordination by the conjugate, (iii) a carrier solution, and (iv) instructions for their combination and use. Depending on the metal, a reducing agent may be necessary to prepare the metal for reaction with the conjugate. Exemplary reducing agents include Ce (III), Fe (II), Cu (I), Ti (III), Sb (III), and Sn (II). Of these, Sn (II) is particularly preferred. Often the components of the kit are in unit dosage form (e.g., each component in a separate vial).

For reasons of stability, it may be preferred that the conjugate be provided in a dry, lyophilized state. The user may then reconstitute the conjugate by adding the carrier or other solution.

Because of the short half-life of suitable radionuclides, it will frequently be most convenient to provide the kit to the user without a radionuclide. The radionuclide is then ordered separately when needed for a procedure. Alternatively, if the radionuclide is included in the kit, the kit will most likely be shipped to the user just before it is needed.

In addition to the metal coordinating moiety, biomolecule, active urea, metal and deprotecting acid, the kit of the present invention typically includes a buffer. Exemplary buffers include citrate, phosphate and borate.

The kit optionally contains other components frequently intended to improve the ease of synthesis of the radiopharmaceutical by the practicing end user, the ease of manufacturing the kit, the shelf-life of the kit, or the stability and shelf-life of the radiopharmaceutical. Such components of the present invention include lyophilization aids, e.g., mannitol, lactose, sorbitol, dextran, Ficoll, and polyvinylpyyrolidine (PVP); stabilization aids, e.g., ascorbic acid, cysteine, monothioglycerol, sodium bisulfite, sodium metabisulfite, gentisic acid, and inositol; and bacteriostats, e.g., benzyl alcohol, benzalkonium chloride, chlorbutanol, and methyl, propyl, or butyl paraben.

Typically, when the conjugate is formulated as a kit, the kit includes multiple vials consisting of a protected metal coordinating moiety having an active urea group, a deprotecting acid, a buffer, and a solution of a radioactive metal such as, but not limited to, In-111, Y-90 or Lu-177. In practice, the user will take the vial containing the metal coordinating moiety and add a solution of a bio-directing carrier of interest bearing a reactive amino (NH₂) group. Once conjugation is complete, the deprotecting acid is added to affect deprotection, followed by addition of the radioactive metal. The mixture is then buffered to complete complexation of the radioactive metal by the metal chelator.

### DEFINITIONS

The compounds described herein may have asymmetric centers. Compounds of the present invention containing an asymmetrically substituted atom may be isolated in optically active or racemic form. Cis and trans geometric isomers of the compounds of the present invention are described and may be isolated as a mixture of isomers or as separated isomeric forms. All chiral, diastereomeric, racemic forms and all geometric isomeric forms of a structure are intended, unless the specific stereochemistry or isomeric form is specifically indicated. All processes used to prepare compounds of the present invention and intermediates made therein are considered to be part of the present invention.

The present invention includes all isotopes of atoms occurring in the present compounds. Isotopes include those atoms having the same atomic number but different mass numbers.

Unless otherwise indicated, the alkyl groups described herein are preferably lower alkyl containing from one to eight carbon atoms in the principal chain and up to 20 carbon atoms. They may be straight or branched chain or cyclic and include methyl, ethyl, propyl, isopropyl, butyl, hexyl and the like.

The term “amido” as used herein includes substituted amido moieties where the substituents include, but are not limited to, one or more of aryl and C₁₋₂₀ alkyl, each of which may be optionally substituted by one or more aryl, carbaldehyde, keto, carboxyl, cyano, halo, nitro, C₁₋₂₀ alkyl, sulfato, sulfito, phosphato, phosphito, hydroxyl, oxy, mercapto, and thio substituents.

The term “amino” as used herein includes substituted amino moieties where the substituents include, but are not limited to, one or more of aryl and C₁₋₂₀ alkyl, each of which may be optionally substituted by one or more aryl, carbaldehyde, keto, carboxyl, cyano, halo, nitro, C₁₋₂₀ alkyl, sulfato, sulfito, phosphato, phosphito, hydroxyl, oxy, mercapto, and thio substituents.

The terms “aryl” or “ar” as used herein alone or as part of another group denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “complex” refers to a metal coordinating moiety of the invention, e.g. Formula (1), complexed or coordinated with a metal. The metal is typically a radioactive isotope or paramagnetic metal ion.

The term “conjugate” refers to a metal coordinating moiety of the invention, e.g. Formula (1), bonded to a bio-directing carrier (biomolecule) whether or not the metal coordinating moiety is complexed with a metal. For the present invention, the metal coordinating moiety is bonded to the bio-directing carrier directly or indirectly by a urea moiety.

The terms “halogen” or “halo” as used herein alone or as part of another group refer to chlorine, bromine, fluorine, and iodine.

The term “heteroatom” shall mean atoms other than carbon and hydrogen.

The terms “heterocyclo” or “heterocyclic” as used herein alone or as part of another group denote optionally substituted, fully saturated or unsaturated, monocyclic or bicyclic, aromatic or nonaromatic groups having at least one heteroatom in at least one ring. The heterocyclo group preferably has 1 to 5 nitrogen atoms in the ring, and may be bonded to the remainder of the molecule through a carbon atom. Exemplary heterocyclics include macrocyclics, cyclen, tacn, DOTA, DOTMA, DOTP, and TETA.

The “heterosubstituted alkyl” moieties described herein are alkyl groups in which a carbon atom is covalently bonded to at least one heteroatom and optionally with hydrogen, the heteroatom being, for example, a nitrogen atom.

The term “metallopharmaceutical” as used herein refers to a pharmaceutically acceptable compound including a metal, wherein the compound is useful for imaging or treatment.

1. A conjugate comprising a selective COX-2 targeting carrier, a metal coordinating moiety, and a linker chemically linking the metal coordinating moiety to the carrier.

2. The conjugate of claim 1, wherein the conjugate has the formula: 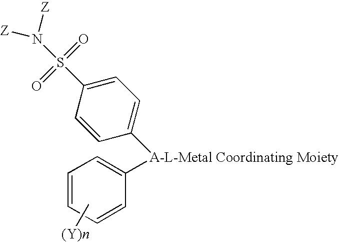
wherein
A is a five- or six-membered ring;
Metal Coordinating Moiety is a moiety that coordinates a radioisotope or paramagnetic metal under physiological conditions;
L is a linker, covalently linking the moiety A, to the Metal Coordinating Moiety;
each Z is independently H, lower alkyl, hydroxyl, hydroxylalkyl, and halo;
each Y is independently H, lower alkyl, hydroxyl, alkyloxy, halo, haloalkyl, amino, aminoalkyl, and phenyl; and
n is 0-3.

3. The conjugate of claim 1, wherein the conjugate has the formula: 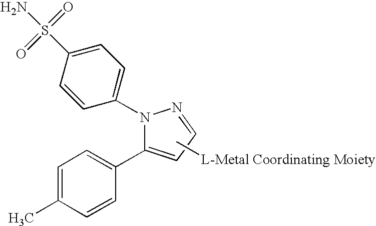
wherein
Metal Coordinating Moiety is a moiety that coordinates a radioisotope or paramagnetic metal under physiological conditions; and
L is a linker, covalently linking the selective COX-2 targeting carrier to the Metal Coordinating Moiety.

4. The conjugate of claim 1, wherein the conjugate has the formula: 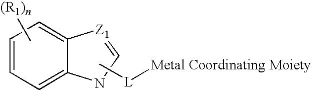
wherein
R₁ is selected from the group consisting of lower alkyl; alkoxy; halo; haloalkoxy;
and haloalkyl;
n is 0-3;
Z₁ is carbon or nitrogen;
Metal Coordinating Moiety is a moiety that coordinates a radioisotope or paramagnetic metal under physiological conditions; and
L is a linker, covalently linking the selective COX-2 targeting carrier to the Metal Coordinating Moiety.

5. The conjugate of claim 1, wherein the conjugate has the formula: 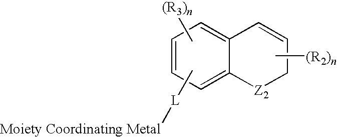
wherein
R₂ is selected from the group consisting of H; lower alkyl; halo; haloalkyl; alkylthio; alkoxy; arylalkyl; cycloalkyl; phenyl; and alkylsulfonyl;
R₃ is selected from the group consisting of H; lower alkyl; haloalkyl; alkoxy; alkylamino; aryl; arylalkyl; aryloxy; arylamino; nitro; sulfonamide; and carboxamide;
n is 2-3;
Z₂ is selected from the group consisting of O, S, NR₄, and CR₅R₆, wherein
R₄ is selected from the group consisting of H; lower-alkyl; aryl; alkylcarboxylic acid; arylcarboxylic acid; alkylsulfonyl; arylsulfinyl; arylsulfonyl; and sulfonamide;
R₅ and R₆ are each independently H; lower alkyl; lower alkyl-phenyl; haloalkyl; halo; or alkenyl;
Metal Coordinating Moiety is a moiety that coordinates a radioisotope or paramagnetic metal under physiological conditions; and
L is a linker, covalently linking the moiety A, to the Metal Coordinating Moiety.

6. The conjugate of claim 1, wherein the COX-2 targeting carrier comprises a derivative of a COX-2 inhibitor selected from the group consisting of celecoxib; cimicoxib; deracoxib; valdecoxib; rofecoxib; etoricoxib; meloxicam; parecoxib; 4-(4-cyclohexyl-2-methyloxazol-5-yl)-2-fluorobenzenesulfonamide; difluorophenyl)-3-(4-(methylsulfonyl)phenyl)-2-cyclopentene-1-one; N-[2-(cyclohexyloxy)-4-nitrophenyl]methanesulfonamide; 2-(3,4-difluorophenyl)-4-(3-hydroxy-3-methylbutoxy)-5-[4-(methylsulfonyl)phenyl]-3(2H)-pyridazinone; 2-[(2,4-dichloro-6-methylphenypamino]-5-ethyl-benzeneacetic acid; (3Z)-3-[(4-chlorophenyl)[4-(methylsulfonyl)phenyl]methylene]-dihydro-2(3H)-furanone; (S)-6,8-dichloro-2-(trifluoromethyl)-2H-1-benzopyran-3-carboxylic acid; lumiracoxib; and any pharmaceutically acceptable salts, esters, or prodrugs thereof.

7. The conjugate of claim 1, wherein the metal coordinating moiety is selected from the group consisting of diacetic amine, DTPA, EDTA, DCTA, DOTA, NOTA, TETA, or analogs or homologs thereof.

8. (canceled)

9. The conjugate of claim 1 wherein the metal coordinating moiety comprises a substituted heterocyclic ring having the following structure: 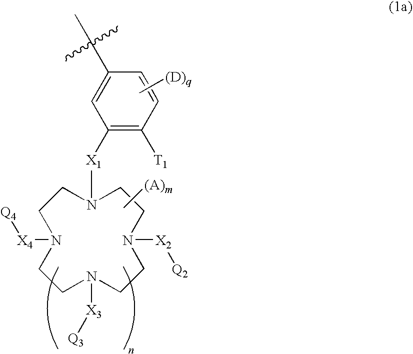
wherein
n is 0, 1 or 2;
m is 0-16 wherein when m is greater than 0, each A is C₁₋₂₀ alkyl or aryl optionally substituted by one or more aryl, C₁₋₂₀ alkyl, carbaldehyde, keto, carboxyl, cyano, halo, nitro, amido, sulfato, sulfito, phosphato, phosphito, hydroxyl, oxy, mercapto or thio;
q is 0-3 wherein when q is greater than 0, each D is independently selected from the group consisting of fluoro, chloro, bromo, iodo, carboxyl, cyano, nitro, amido, hydroxyl, amino, sulfato, sulfito, phosphato, phosphito, aryl, and C₁₋₂₀ alkyl optionally substituted with one or more of C₁₋₂₀ alkyl, carboxyl, cyano, nitro, amido, hydroxyl, amino, sulfato, sulfito, phosphato, and phosphito;
X₁, X₂, X₃, X₄ are independently optionally substituted methylene where the substituents are selected from the group consisting of aryl, C₁₋₂₀ alkyl, carbaldehyde, keto, carboxyl, cyano, halo, nitro, amido, sulfato, sulfito, phosphato, phosphito, hydroxyl, oxy, mercapto and thio;
Q₂-Q₄ are independently selected from the group consisting of: 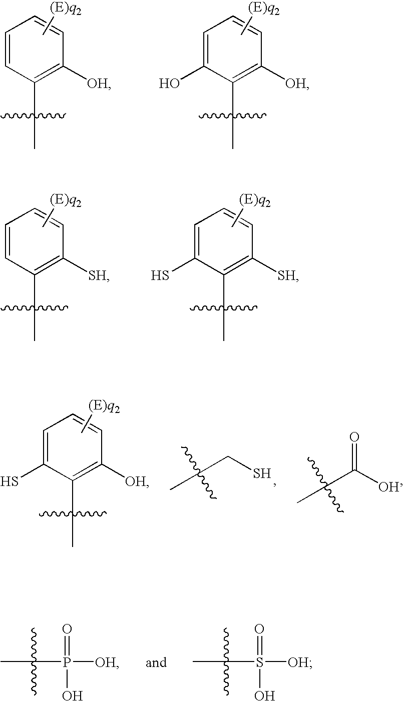
q₂ is 0-4 wherein when q₂ is greater than 0, each E is independently selected from the group consisting of fluoro, chloro, bromo, iodo, carboxyl, cyano, nitro, amido, hydroxyl, amino, sulfito, phosphito, and C₁₋₂₀ alkyl optionally substituted with one or more or C₁₋₂₀ alkyl, carboxy, cyano, nitro, amido, hydroxyl, sulfito, phospito, sulfate, and phosphato; and
T₁ is hydroxyl or mercapto.

10-12. (canceled)

13. The conjugate of claim 1, wherein the metal coordinating moiety is complexed with a metal, the metal consisting of a radioisotope or a paramagnetic metal.

14. The conjugate of claim 13, wherein the metal is selected from the group consisting of Cr(III), Mn(II), Fe(III), Fe(II), Co(II), Ni(II), Cu(II), Nd(III), Sm(III), Y(III), Gd(III), V(II), Tb(III), Dy(III), Ho(III), Er(III), Cu, Cu-62, Cu-64, Cu-67, Ga, Ga-67, Ga-68, As, As-77, Y, Y-86, Zr-89, Y-90, Tc, Tc═O, Tc-94, Tc-94m, Tc-99m, Tc-99m=O, Pd, Pd-103, In, In-111, Ag-111, I-123, I-124, I-125, I-131, Pr-142, Pm, Pm-149, Gd, Gd-153, Sm, Sm-153, Tb-161, Dy, Dy-165, Dy-166, Ho, Ho-166, Eu, Eu-169, Tm, Tm-170, Lu, Lu-177, Re, Re-186, Re-188, Re═O, Re-186=O, Re-188=O, At, At-211, Bi, Bi-212, Bi-212, Bi-213, Pb-212, Ra-223, and Ac-225.

15-17. (canceled)

18. A pharmaceutical composition comprising the conjugate of claim 1 and a pharmaceutically acceptable carrier.

19. A method of diagnosing or treating a disease associated with the over-expression of COX-2, the method comprising:
administering to a patient an amount of a conjugate comprising a selective COX-2 targeting carrier linked to a metal coordinating moiety chelating a radioisotope or paramagnetic metal under physiological conditions, said selective COX-2 targeting carrier binding to a site of COX-2 over-expression.

20-27. (canceled)

28. The method of claim 19, wherein the metal coordinating moiety is complexed with Tc-99m, the conjugate having the formula:

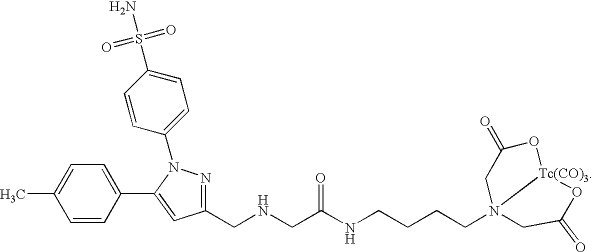

29. The method of claim 19 wherein the conjugate has the formula:

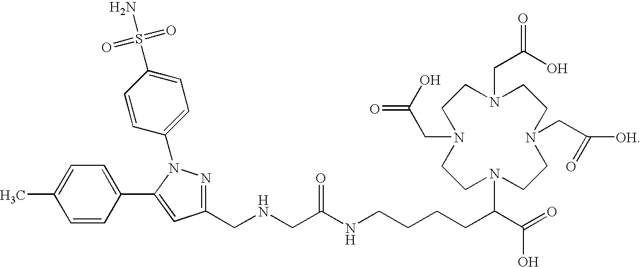

30. The method of claim 19, wherein the metal coordinating moiety is complexed with Re-188, the conjugate having the formula:

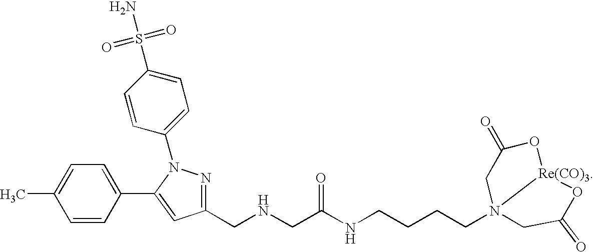

31-43. (canceled)

44. A method of treating a tumor associated with the expression of prostaglandins, the method comprising:
administering to a patient an amount of a conjugate comprising a selective COX-2 targeting carrier linked to a metal coordinating moiety chelating a radioisotope, said selective COX-2 targeting carrier binding to a tumor site and reducing the expression of COX-2-derived prostaglandins,
wherein reduction of the tumor size following administration of the conjugate is greater than the reduction of tumor size following the administration a combination therapy of a similar dose of a COX-2 inhibitor monomer corresponding to the COX-2 targeting carrier and a similar dose of external radiotherapy.

45. The method of 44, wherein the COX-2-derived prostaglandins are selected from the group consisting of prostaglandin E₂, prostaglandin Fₐₕ 6-Keto-prostaglandin F_{1α}, and thromboxane B₂.

46-49. (canceled)

50. The method of claim 44, wherein the wherein the conjugate has the formula: 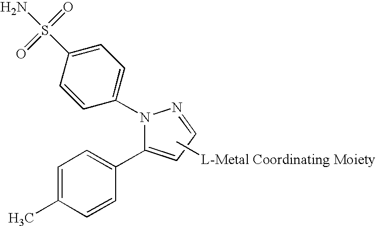
wherein
Metal Coordinating Moiety is a moiety that coordinates a radioisotope or paramagnetic metal under physiological conditions; and
L is a linker, covalently linking the selective COX-2 targeting carrier to the Metal Coordinating Moiety.

51. The method of claim 50, wherein the conjugate is selected from:

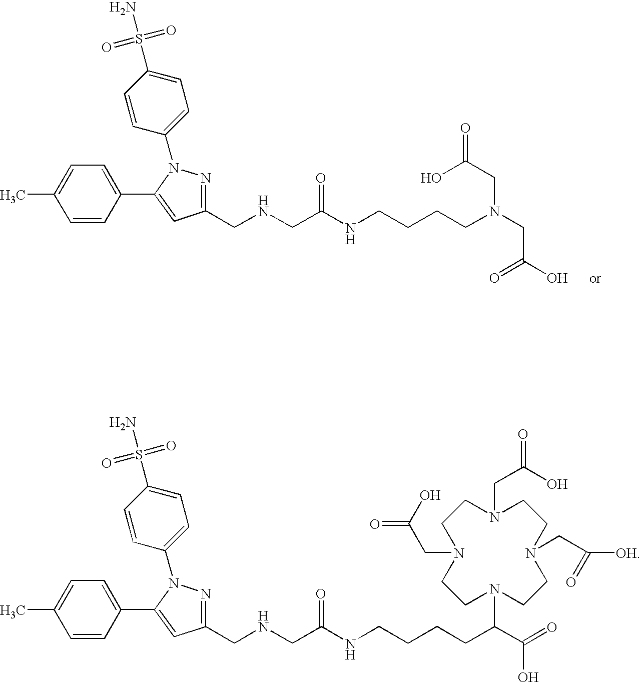

52. The method of claim 44, wherein the radioisotope is selected from the group consisting of Cu-64, Cu-67, Ga-67, Y-90, Ag-111, In-111, I-123, I-131, Pr-142, Sm-153, Tb-161, Dy-166, Ho-166, Lu-177, Re-186, Re-188, Re-189, At-211, Pb-212, Bi-212, Bi-213, Ra-223, and Ac-225.

53-55. (canceled)
